(19) United States
(12) Patent Application Publication
(10) Pub. No.: US 2024/0416328 A1
(43) Pub. Date: Dec. 19, 2024

(54) METHOD FOR PRODUCING CATALYST MOLDED ARTICLE USED WHEN PRODUCING alpha,beta-UNSATURATED CARBOXYLIC ACID, AND METHOD FOR PRODUCING alpha,beta-UNSATURATED CARBOXYLIC ACID AND alpha,beta-UNSATURATED CARBOXYLIC ACID ESTER USING SAME

(71) Applicant: MITSUBISHI CHEMICAL CORPORATION, Tokyo (JP)

(72) Inventors: Akio TAKEDA, Tokyo (JP); Takuro WATANABE, Tokyo (JP); Yuki KATO, Tokyo (JP)

(73) Assignee: MITSUBISHI CHEMICAL CORPORATION, Tokyo (JP)

(21) Appl. No.: 18/814,026

(22) Filed: Aug. 23, 2024

Related U.S. Application Data
(63) Continuation of application No. PCT/JP2023/005087, filed on Feb. 15, 2023.

(30) Foreign Application Priority Data
Feb. 25, 2022 (JP) 2022-028159

(51) Int. Cl. B01J 27/199; B01J 37/06; B30B 11/02; C07C 51/16; C07C 67/04

(52) U.S. Cl. CPC B01J 27/199; B01J 37/06; C07C 51/16; C07C 67/04; B30B 11/02

(57) ABSTRACT

An object is to provide a method capable of stably producing a catalyst molded article which is favorable in moldability in a step of molding a dried catalyst product and which provides a high yield of α,β-unsaturated carboxylic acid, and methods for producing α,β-unsaturated carboxylic acid and α,β-unsaturated carboxylic acid ester with the catalyst molded article. The object is achieved by a method for producing a catalyst molded article by preparing a catalyst raw material-containing slurry with a catalyst production apparatus washed under specified conditions, and methods for producing α,β-unsaturated carboxylic acid and α,β-unsaturated carboxylic acid ester with the catalyst molded article.

### CROSS-REFERENCE TO RELATED APPLICATION

This is a continuation of International Application PCT/JP2023/005087, filed on Feb. 15, 2023, and designated the U.S., and claims priority from Japanese Patent Application 2022-028159 which was filed on Feb. 25, 2022, the entire contents of which are incorporated herein by reference.

### TECHNICAL FIELD

The present invention relates to a method for producing a catalyst molded article for use in the production of α,β-unsaturated carboxylic acid, and methods for producing α,β-unsaturated carboxylic acid and α,β-unsaturated carboxylic acid ester with the catalyst molded article.

### BACKGROUND ART

There are known as catalysts for α,β-unsaturated carboxylic acid production, for use in the production of α,β-unsaturated carboxylic acid by oxidation of α,β-unsaturated aldehyde (hereinafter, also simply referred to as “catalysts”), catalysts containing alkali metal salts of heteropoly acid including phosphorus and molybdenum. Alkali metal salts of heteropoly acid have cations large in ionic radii, and are commonly poorly soluble (NPL 1).

In a case where catalysts contain poorly-soluble salts such as alkali metal salts of heteropoly acid, catalysts are industrially repeatedly produced and thus cause attachment of solids due to catalyst raw materials, onto surfaces of catalyst production apparatuses. Examples of methods for suppressing the influence of such solids on the quality of catalysts include a method described in PTL 1, in which catalyst raw material slurries are obtained in at least two different formulation vessels and thereafter these catalyst raw material slurries are mixed by use of one mixing vessel different from the formulation vessels. Thus, substances different in structure and composition from objective catalysts can be inhibited from being generated in steps of producing catalyst raw material-containing slurries.

Catalysts for α,β-unsaturated carboxylic acid production are commonly utilized for reaction, as catalyst molded articles obtained by molding dried catalyst products of catalyst raw material-containing slurries into spherical products each having a diameter of about 2 to 20 mm, or columnar or cylinder products each having a diameter of about 2 to 10 mm and a length of about 2 to 20 mm. Examples of methods for producing catalyst molded articles include a method described in PTL 2, the method including a primary molding step of primarily molding a kneaded product obtained by kneading a dried catalyst product and a liquid, and a secondary molding step of extruding a primarily molded article into a final shape by a piston molding machine, in which the molding pressure is prescribed.

### RELATED ART DOCUMENTS

### Patent Documents

PTL 1: WO 2015/037611

PTL 2: JP-A 2011-224482

### NON PATENT LITERATURE

NPL 1: Otake Masayuki, Onoda Takeshi, Catalysis Society of Japan, “Catalyst”, vol. 18, No. 6 (1976), p. 169

### SUMMARY OF THE INVENTION

There have been studied methods for reducing the influence of solids in step of producing catalyst raw material-containing slurries in the case of repeated production of catalysts, as described above. However, there has not been studied heretofore any influence of solids in the steps of molding dried catalyst products to produce catalyst molded articles. The present inventors have found that the presence of solids has an adverse influence on moldability in steps of molding dried catalyst products in the production of catalyst molded articles for use in the production of α,β-unsaturated carboxylic acid, and thus have completed the present invention.

An object of the present invention is to provide a method capable of stably producing a catalyst molded article which is favorable in moldability in a step of molding a dried catalyst product and which provides a high yield of α,β-unsaturated carboxylic acid.

The present inventors have made intensive studies in view of the above problems, and as a result, have found that the above problems can be solved by preparing a catalyst raw material-containing slurry with a catalyst production apparatus washed under specified conditions, and thus have completed the present invention.

Specifically, the present invention includes the following aspects.
[1]: A method for producing a catalyst molded article for use in the production of α,β-unsaturated carboxylic acid, by oxidation of α,β-unsaturated aldehyde, the method including
   (i) a step of washing a surface of a catalyst production apparatus where a solid including phosphorus, molybdenum and an alkali metal is attached onto the surface, with a basic solution,
   (ii) a step of using the catalyst production apparatus, to prepare a slurry A containing a heteropoly acid salt including phosphorus and molybdenum,
   (iii) a step of drying the slurry A, to obtain a dried catalyst product, and
   (iv) a step of molding the dried catalyst product, to obtain a catalyst molded article, wherein the basic solution after washing the surface of the catalyst production apparatus in step (i) has a pH of 9 or more.
[2]: The method for producing a catalyst molded article according to [1], wherein the dried catalyst product is molded with an extrusion molding machine in step (iv).
[3]: The method for producing a catalyst molded article according to [2], wherein the dried catalyst product is molded at an extrusion rate of 50 to 500 mm/min in step (iv).
[4]: The method for producing a catalyst molded article according to [2] or [3], wherein the dried catalyst product is molded at an extrusion rate of 50 to 300 mm/min in step (iv).
[5]: The method for producing a catalyst molded article according to any of [2] to [4], wherein the maximum value of the pressure in the extrusion molding machine in step (iv) is 15 MPa or less in terms of gauge pressure.
[6]: The method for producing a catalyst molded article according to any of [1] to [5], wherein the surface of the catalyst production apparatus is washed with the basic solution being stirred at a power required for stirring per unit volume of 0.01 to 6 kW/m3 in step (i).
[7]: The method for producing a catalyst molded article according to any of [1] to [6], wherein the surface of the catalyst production apparatus is washed with the basic solution being circulated in a pipe connected to the catalyst production apparatus, by use of a circulation pump, in step (i).
[8]: The method for producing a catalyst molded article according to [7], wherein the linear velocity of the basic solution circulated in the pipe is 1 to 100 cm/s.
[9]: The method for producing a catalyst molded article according to any of [1] to [8], wherein the dried catalyst product has a composition represented by the following formula (I):

PₐMO_{b}V_{c}CU_{d}AₑE_{f}G_{g}(NH₄)ₕOᵢ (I)

wherein P, Mo, V, Cu, NH₄ and 0 respectively represent phosphorus, molybdenum, vanadium, copper, ammonium radical and oxygen, A represents at least one element selected from the group consisting of antimony, bismuth, arsenicum, germanium, tellurium, selenium, silicon and tungsten, E represents at least one element selected from the group consisting of iron, zinc, chromium, tantalum, cobalt, nickel, manganese, titanium and niobium, G represents at least one element selected from the group consisting of potassium, rubidium and cesium, a to i each represent the molar ratio of each component, b=12, a=0.5 to 3, c=0.01 to 3, d=0.01 to 2, e=0 to 3, f=0 to 3, g=0.01 to 3, and h=0 to 20 are satisfied, and i is the molar ratio of oxygen necessary for satisfying the valence of such each component.
[10]: The method for producing a catalyst molded article according to any of [1] to [9], wherein the dried catalyst product has a Keggin-type heteropoly acid structure.
[11]: The method for producing a catalyst molded article according to any of [1] to [10], including a step of washing the surface of the catalyst production apparatus, washed with the basic solution in step (i), with water, to remove the basic solution.
[12]: The method for producing a catalyst molded article according to [11], wherein the basic solution is removed by washing with stirring of water at a power required for stirring per unit volume of 0.01 to 6 kW/m3.
[13]: A method for producing α,β-unsaturated carboxylic acid, including oxidizing α,β-unsaturated aldehyde in the presence of a catalyst molded article produced by the method according to any of [1] to [12].
[14]: A method for producing α,β-unsaturated carboxylic acid ester, including esterifying α,β-unsaturated carboxylic acid produced by the method according to.

### Effects of the Invention

According to the present invention, there can be provided a method capable of stably producing a catalyst molded article which is favorable in moldability and which provides a high yield of α,β-unsaturated carboxylic acid, and methods for producing α,β-unsaturated carboxylic acid and α,β-unsaturated carboxylic acid ester with the catalyst molded article.

### Mode for Carrying Out the Invention

Hereinafter, embodiments of the present invention are described in detail, but the description of constituent requirements described below is illustrative for carrying out aspects of the present invention, and the present invention is not identified by these contents.

Herein, a numerical value range expressed with “to” means a range including numerical values described before and after “to” respectively as the lower limit value and the upper limit value, and “A to B” means A or more and B or less.

### (Method for Producing Catalyst Molded Article)

A first embodiment of the present invention provides a method for producing a catalyst molded article for use in the production of α,β-unsaturated carboxylic acid, by oxidation of α,β-unsaturated aldehyde, the method including the following steps (i) to (iv).
(i) A step of washing a surface of a catalyst production apparatus where a solid including phosphorus, molybdenum and an alkali metal is attached onto the surface, with a basic solution.
(ii) A step of using the catalyst production apparatus, to prepare a slurry A containing a heteropoly acid salt including phosphorus and molybdenum.
(iii) A step of drying the slurry A, to obtain a dried catalyst product.
(iv) A step of molding the dried catalyst product, to obtain a catalyst molded article.

Herein, the basic solution after washing the surface of the catalyst production apparatus in step (i) has a pH of 9 or more.

The production method can be used to stably obtain a catalyst molded article which is favorable in moldability and which provides a high yield of α,β-unsaturated carboxylic acid.

Hereinafter, each step is described in detail.

<Step (i)>

In step (i), a surface of a catalyst production apparatus where a solid including phosphorus, molybdenum and an alkali metal is attached onto the surface is washed with a basic solution. The pH of the basic solution after washing the surface of the catalyst production apparatus is 9 or more. The “pH” herein is a value measured with a pH meter under conditions of 25° C. and 1 atm. The pH meter here used can be a commercially available pH meter, and can be, for example, a pH meter such as D-21 (manufactured by Horiba Ltd.).

### (Surface of Catalyst Production Apparatus)

The surface of the catalyst production apparatus means a surface with which a slurry A containing a heteropoly acid salt including phosphorus and molybdenum is to be contacted in step (ii) described below. Examples include an inner wall of a vessel unit for preparation of the slurry A, a surface of an interior object such as a stirring apparatus or a heater used in a vessel unit, an inner surface of ancillary equipment pertaining to a vessel unit, such as a strainer or a pump, and an inner wall of a pipe connected to a vessel unit.

### (Solid)

The solid attached onto the surface of the catalyst production apparatus is a solid which is attached by repeated catalyst production and which is due to a catalyst raw material, the solid including phosphorus, molybdenum and an alkali metal. Such inclusion of phosphorus, molybdenum and an alkali metal in the solid can be confirmed by dissolving the solid in ammonia water and analyzing each dissolved component with ICP emission spectroscopy. The analysis apparatus here used can be, for example, ICP Optima 8300 (manufactured by Perkin Elmer).

### (Preliminary Washing)

The surface of the catalyst production apparatus may be washed with a solvent in advance before washing with the basic solution (hereinafter, also referred to as “preliminary washing”). Thus, the amount of the basic solution used can be reduced in washing with the basic solution.

The preliminary washing may be performed for all or only one portion of the surface of the above catalyst production apparatus. The preliminary washing is preferably performed for such a surface of the catalyst production apparatus, including a portion to be washed with the basic solution, from the viewpoint of a reduction in amount of the basic solution used. The preliminary washing is here more preferably performed also for a surface of an interior object such as a stirring apparatus or a heater used in a vessel unit, and an inner wall of a pipe connected to the vessel unit.

The solvent used in the preliminary washing is not particularly limited as long as it can release or dissolve one portion of the solid, and is preferably water.

The method of the preliminary washing is not particularly limited as long as it is a method including contacting the solvent with the solid. Examples of the case of washing a vessel unit include a method including loading the solvent so that the solvent is contacted with the inner wall of the vessel unit, and allowing the solvent to still stand or to flow, and a method including allowing the solvent to flow is preferably used. Examples of the method including allowing the solvent to flow include a method including stirring the solvent with a stirring apparatus, a method including circulating the solvent with a circulation pump, and a method as combination use of these methods. Herein, the “circulation” means circulation in a pipe connected to the catalyst production apparatus.

The preliminary washing is preferably performed at a temperature of 5 to 80° C. from the viewpoint of the washing effect, and the lower limit is more preferably 10° C. or more and the upper limit is more preferably 70° C. or less.

### (Washing)

The washing with the basic solution may be performed for all or only one portion of the surface of the above catalyst production apparatus. The washing is preferably performed for such a surface of the catalyst production apparatus, including an inner wall of a vessel unit for preparation of the slurry A, from the viewpoint of moldability in step (iv) described below. The washing is here more preferably performed also for a surface of an interior object such as a stirring apparatus or a heater used in the vessel unit, and an inner wall of a pipe connected to the vessel unit.

The basic solution for washing the surface of the catalyst production apparatus is not particularly limited as long as it is obtained by dissolving a basic substance in a solvent. The concentration of the basic substance in the basic solution is preferably 1 to 10% by mass, and the lower limit is more preferably 2% by mass or more and the upper limit is more preferably 6% by mass or less. Examples of the basic substance include oxide, hydroxide, carbonate and hydrogencarbonate of an alkali metal, and oxide and hydroxide of an alkali earth metal. The basic substance is preferably at least one selected from the group consisting of oxide, hydroxide, carbonate and hydrogencarbonate of an alkali metal, particularly preferably hydroxide of an alkali metal. Examples of the alkali metal include lithium, sodium, potassium and rubidium, and potassium is preferred. The basic substance may be used singly or in combination of two or more kinds thereof. Examples of the solvent of the basic solution include water and alcohol, and water is preferred.

The basic solution is preferably used in an amount of 0.5 to 1 time the volume of the catalyst production apparatus, and the lower limit is preferably an amount of 0.8 times or more and the upper limit is preferably an amount of 0.98 times or less.

The method of washing with the basic solution is not particularly limited as long as it is a method including contacting the basic solution with the solid.

Examples of the case of washing a vessel unit include a method including loading the basic solution so that the basic solution is contacted with the inner wall of the vessel unit, and allowing the basic solution to still stand or to flow, and a method including allowing the basic solution to flow is preferably used. Examples of the method including allowing the basic solution to flow include a method including stirring the basic solution with a stirring apparatus, a method including circulating the basic solution with a circulation pump, and a method as combination use of these methods. A method including stirring the basic solution with a stirring apparatus is preferably used, and a method including stirring the basic solution with a stirring apparatus and a method including circulating the basic solution with a circulation pump are more preferably used, from the viewpoint of moldability in step (iv) described below. In a case where an inner wall of a pipe connected to the catalyst production apparatus is also washed, a method is preferred in which, while the basic solution is circulated in the pipe connected to the catalyst production apparatus with a circulation pump, the surface of the catalyst production apparatus is washed.

The basic solution is preferably stirred at a power required for stirring per unit volume of 0.01 to 6 kW/m³. The lower limit of the power required for stirring per unit volume is more preferably 0.05 kW/m³ or more, further preferably 0.1 kW/m³ or more, particularly preferably 0.5 kW/m³ or more, most preferably 1 kW/m³ or more. The upper limit is more preferably 5.5 kW/m³ or less, further preferably 5 kW/m³ or less.

In a case where a basic solution is circulated with a circulation pump, the linear velocity of the basic solution circulated in the pipe is preferably 1 to 100 cm/s. The lower limit of the linear velocity of the basic solution circulated in the pipe is more preferably 3 cm/s or more, further preferably 5 cm/s or more. The upper limit is more preferably 80 cm/s or less, further preferably 50 cm/s or less, particularly preferably 30 cm/s or less.

The washing time is not particularly limited and any time can be selected depending on the type and amount of the solid, the type and amount of the basic solution, and the like, as long as the solid can be decreased. Such a decrease of the solid by the washing can be confirmed by visually observing the surface of the catalyst production apparatus washed. The washing time is generally 0.1 to 10 hours, and the lower limit is preferably 1 hour or more and the upper limit is preferably 5 hours or less.

The washing can be performed at any temperature in consideration of, for example, the degree of dissolution of the solid, such any temperature is preferably 5 to 80° C. from the viewpoint of the washing effect, and the lower limit is more preferably 10° C. or more and the upper limit is more preferably 70° C. or less.

The washing may be performed under an increased pressure of more than the atmospheric pressure, under the atmospheric pressure, or under a reduced pressure of less than the atmospheric pressure. The washing is preferably under a reduced pressure from the viewpoint of, for example, suction of vapor generated in the washing, and is more preferably performed under a condition so that the pressure in the catalyst production apparatus is-0.05 to −0.001 MPa (G). Herein, (G) means the gauge pressure.

When the catalyst molded article for use in the production of α,β-unsaturated carboxylic acid is produced, the presence of the solid attached onto the surface of the catalyst production apparatus has an adverse influence on moldability in step (iv) described below. The solid reacts with the basic substance included in the basic solution to form a neutralized salt, and the neutralized salt is released or dissolved in the basic solution. Accordingly, in a case where a large amount of such a solid not forming any neutralized salt remains after the washing, namely, in a case where all of the basic substance included in the basic solution forms a neutralized salt, the pH is neutral or lower. Thus, the amount of the remaining solid attached onto the surface of the catalyst production apparatus can be determined from the pH of the basic solution after the washing. In the present embodiment, the basic solution after washing the surface of the catalyst production apparatus in step (i) has a pH of 9 or more. This indicates that the solid attached onto the surface of the catalyst production apparatus is sufficiently decreased. Thus, favorable moldability is exhibited in step (iv) described below. The lower limit of the pH of the basic solution after the washing is preferably 9 or more, more preferably 9.5 or more, further preferably 10 or more. The upper limit of the pH of the basic solution after the washing is not particularly restricted, and is usually 12 or less. The same also applies to a preferred range of the pH of the basic solution before the washing.

The washing with the basic solution may be performed once, or twice or more. In a case where the washing is performed twice or more, the basic solution used in the washing may be removed and a fresh basic solution may be used, or the basic solution used in the washing may be partially or fully repeatedly used. In the present embodiment, in a case where the washing is performed twice or more, the pH of the basic solution after any such washing may be 9 or more.

### (Subsequent Washing)

The surface of the catalyst production apparatus may be further washed with a solvent after washing with the basic solution, to remove the basic solution (hereinafter, also referred to as “subsequent washing”). Thus, the influence of the basic substance remaining on the surface of the catalyst production apparatus, on formation of a heteropoly acid salt, in step (ii) described below can be reduced.

The subsequent washing may be performed for all or only one portion of the surface of the above catalyst production apparatus. The subsequent washing is preferably performed for such a surface of the catalyst production apparatus, including a portion washed with the basic solution, from the viewpoint of a reduction in influence of the basic substance on formation of a heteropoly acid salt. The subsequent washing is here more preferably performed also for a surface of an interior object such as a stirring apparatus or a heater used in a vessel unit, and an inner wall of a pipe connected to the vessel unit.

The solvent used in the subsequent washing is not particularly limited as long as it can remove the basic substance, and is preferably water. In a case where water is used as the solvent, the pH of such water after the subsequent washing is preferably 8.5 or less, more preferably 6 to 8. Thus, the influence of the basic substance remaining on the surface of the catalyst production apparatus, on formation of a heteropoly acid salt, can be sufficiently reduced, and a reduction in yield of α,β-unsaturated carboxylic acid can be suppressed.

The method of the subsequent washing is not particularly limited as long as it is a method including contacting the solvent with the solid. Examples of the case of washing a vessel unit include a method including loading the solvent so that the solvent is contacted with the inner wall of the vessel unit, and allowing the solvent to still stand or to flow, and a method including allowing the solvent to flow is preferably used. Examples of the method including allowing the solvent to flow include a method including stirring the solvent with a stirring apparatus, a method including circulating the solvent with a circulation pump, and a method as combination use of these methods.

The solvent is preferably stirred at a power required for stirring per unit volume of 0.01 to 6 kW/m³. The lower limit of the power required for stirring per unit volume is more preferably 0.05 kW/m³ or more, further preferably 0.1 kW/m³ or more, particularly preferably 0.5 kW/m³ or more, most preferably 1 kW/m³ or more. The upper limit is more preferably 5.5 kW/m³ or less, further preferably 5 kW/m³ or less.

The subsequent washing is preferably performed at a temperature of 5 to 80° C. from the viewpoint of the washing effect, and the lower limit is 10° C. or more and the upper limit is more preferably 70° C. or less.

The subsequent washing may be performed once, or twice or more. In a case where the subsequent washing is performed twice or more, the solvent used in the subsequent washing may be removed and a fresh solvent may be used, or the solvent used in the subsequent washing may be partially or fully repeatedly used. In a case where the subsequent washing is performed twice or more, the pH of the basic solution after any such subsequent washing is preferably 8.5 or less, more preferably 6 to 8.

<Step (ii)>

In step (ii), the catalyst production apparatus is used to prepare a slurry A containing a heteropoly acid salt including phosphorus and molybdenum. The slurry A can be prepared by dissolving or suspending a raw material compound of a catalyst component including phosphorus and molybdenum in a solvent.

### (Molybdenum Raw Material)

Examples of the molybdenum raw material include molybdenum oxides such as molybdenum trioxide, ammonium molybdates such as ammonium paramolybdate and ammonium dimolybdate, molybdic acid, and molybdenum chloride.

### (Phosphorus Raw Material)

Examples of the phosphorus raw material include phosphoric acid, phosphorus pentoxide, or phosphates such as ammonium phosphates (ammonium dihydrogenphosphate, diammonium hydrogenphosphate, and triammonium phosphate) and cesium phosphate.

### (Other Raw Material of Catalyst Component)

Raw materials of elements constituting a composition represented by formula (I) described below are preferably further used in preparation of the slurry A. Such raw materials are not particularly limited, and oxide, sulfate, nitrate, carbonate, hydroxide, an organic acid salt such as acetate, an ammonium salt, halide, oxyacid, an oxyacid salt, an alkali metal salt, or the like can be used singly or in combination of two or more kinds thereof.

Specifically, examples of a vanadium raw material include ammonium vanadate, ammonium metavanadate, vanadium pentoxide, vanadium chloride, and vanadyl oxalate. Examples of a copper raw material include copper sulfate, copper nitrate, copper oxide, copper carbonate, copper acetate, and copper chloride. An ammonium raw material is not particularly limited as long as it is an ammonium-containing compound. Examples include ammonium phosphate and its hydrate, ammonium hydroxide, ammonia water, ammonium nitrate, ammonium carbonate, and ammonium bicarbonate. These raw materials may be used singly or in combination of two or more kinds thereof.

### (Solvent)

The solvent here used can be water, an organic solvent, a mixed solvent of water and an organic solvent, or the like, and water is preferably used from an industrial viewpoint.

### (Mixing of Raw Materials and Solvent)

The above raw materials are mixed by addition of some or all thereof into the solvent. The order of addition of the raw materials is not particularly limited, and such addition is preferably made so that the pH of the solution or slurry is in the range of 0.1 to 6.5, from the viewpoint that a catalyst molded article providing a high yield of α,β-unsaturated carboxylic acid can be obtained. The lower limit of the pH is more preferably 0.5 or more, further preferably 1 or more. The upper limit is more preferably 6 or less, further preferably 3 or less. The pH of the solution or slurry is not needed to be controlled by critical measurement of the pH with titration, and can be adjusted under measurement with a commercially available pH meter or the like. The pH can be adjusted with, if necessary, any acid (sulfuric acid, nitric acid, hydrochloric acid, or the like) or base containing the same ion as those of the raw materials.

The temperature of the solvent during mixing of the raw materials and the solvent is preferably 10 to 60° C. from the viewpoint of avoiding each of the raw materials from unnecessarily reacting, and the upper limit is more preferably 50° C. or less. The raw materials can also be added with temperature rise as long as the temperature is in the above temperature range.

### (Preparation of Slurry A)

The slurry A is prepared by mixing the raw materials and the solvent, and then stirring the mixture with heating. The slurry A contains a heteropoly acid salt including phosphorus and molybdenum.

The heating temperature is not particularly limited, and is preferably 75 to 130° C., more preferably 95 to 130° C. or less. When the heating temperature is 75° C. or more, the rate of generation of the heteropoly acid salt can be sufficiently increased. When the heating temperature is 130° C. or less, evaporation of water in the slurry A can be suppressed. In addition, concentration and/or reflux may be made during heating, depending on the vapor pressure of the solvent used, or heating treatment may be made under a pressurized condition by operation in a closed container.

The rate of temperature rise is not particularly limited, and is preferably 0.8 to 15° C./min. When the rate of temperature rise is 0.8° C./min or more, the time necessary for step (ii) can be shortened. When the rate of temperature rise is 15° C./min or less, temperature rise can be made with usual temperature rise equipment.

The stirring is preferably performed at a power required for stirring per unit volume of 0.01 kW/m³ or more, more preferably 0.05 kW/m³ or more. When the power required for stirring per unit volume is 0.01 kW/m³ or more, local irregularities of the temperature of the slurry A, the component, and the temperature are decreased and a structure suitable for production of α,β-unsaturated carboxylic acid is stably formed. The stirring is usually preferably performed at a power required for stirring per unit volume of 3.5 kW/m³ or less from the viewpoint of production cost.

The pH of the slurry A obtained in step (ii) is preferably 0.1 to 4, and the lower limit is more preferably 0.5 or more and the upper limit is more preferably 2.5 or less. Thus, a dried catalyst product having a Keggin-type heteropoly acid structure suitable for α,β-unsaturated carboxylic acid production can be obtained in step (iii) described below.

Examples of the method for controlling the pH of each of the solution and the slurry include a method including appropriately selecting the amount of each raw material containing a catalyst constituent component and appropriately adding nitric acid, oxalic acid, or the like.

<Step (iii)>

In step (iii), the slurry A obtained in step (ii) is dried, to obtain a dried catalyst product.

Examples of the drying method include known methods such as a drum drying method, a flash drying method, an evaporation drying method, and a spray drying method. In particular, a spray drying method is preferably used because a particulate dried catalyst product is obtained and the shape of the dried catalyst product is a neat spherical shape.

The drying temperature differs depending on the drying method and can usually be 100 to 500° C., the lower limit is preferably 140° C. or more, and the upper limit is preferably 400° C. or less.

The drying is preferably performed so that the content rate of moisture in the resulting dried catalyst product is 4.5% by mass or less, more preferably 0.1 to 4.5% by mass. These conditions are not particularly limited, and can be appropriately selected depending on the desired shape and size of the dried catalyst product.

The resulting dried catalyst product preferably has an elemental composition represented by the following formula (I) from the viewpoint of an enhancement in yield in α,β-unsaturated carboxylic acid production. The catalyst may include a small amount of any element not described in the following formula (I).

PₐMO_{b}V_{c}CU_{d}AₑE_{f}G_{g}(NH₄)ₕOᵢ (I)

In the formula (I), P, Mo, V, Cu, NH₄ and 0 respectively represent phosphorus, molybdenum, vanadium, copper, ammonium radical and oxygen. A represents at least one element selected from the group consisting of antimony, bismuth, arsenic, germanium, tellurium, selenium, silicon and tungsten. E represents at least one element selected from the group consisting of iron, zinc, chromium, tantalum, cobalt, nickel, manganese, titanium, and niobium. G represents at least one element selected from the group consisting of potassium, rubidium and cesium. a to i each represent the molar ratio of each component, b=12, a=0.5 to 3, c=0.01 to 3, d=0.01 to 2, e=0 to 3, f=0 to 3, g=0.01 to 3, and h=0 to 20 are satisfied, and i is the molar ratio of oxygen necessary for satisfying the valence of such each component.

In the formula (I), from the viewpoint of an enhancement in yield of α,β-unsaturated carboxylic acid, the lower limit of a is preferably 0.6 or more, more preferably 0.7 or more. The upper limit of a is preferably 2.5 or less, more preferably 2 or less. The lower limit of c is preferably 0.1 or more, preferably 0.15 or more, further preferably 0.2 or more. The upper limit of c is preferably 2.5 or less, more preferably 2 or less, further preferably 1.5 or less. The lower limit of d is preferably 0.03 or more, more preferably 0.05 or more. The upper limit of d is preferably 2.5 or less, more preferably 2 or less. The lower limit of e is preferably 0.01 or more, more preferably 0.1 or more. The upper limit of e is preferably 2.5 or less, more preferably 2 or less. The upper limit of f is preferably 2 or less, more preferably 1.5 or less, further preferably 1 or less. The lower limit of g is preferably 0.1 or more, more preferably 0.3 or more. The upper limit of g is preferably 2.8 or less, more preferably 2.5 or less. The upper limit of h is preferably 15 or less, more preferably 10 or less.

The molar ratio of each element is a value calculated by analyzing each component of the dried catalyst product dissolved in ammonia water, with ICP emission spectroscopy. The analysis apparatus here used can be, for example, ICP Optima 8300 (manufactured by Perkin Elmer). The molar ratio of the ammonium radical is a value calculated by analyzing the dried catalyst product according to the Kjeldahl method. The ammonium radical herein is a collective term of ammonia (NH₃) capable of being converted into an ammonium ion (NH₄⁺), and ammonium contained in an ammonium-containing compound such as an ammonium salt.

The resulting dried catalyst product preferably has a Keggin-type heteropoly acid structure from the viewpoint of an enhancement in yield in α,β-unsaturated carboxylic acid production. Examples of the method for obtaining the dried catalyst product having a Keggin-type heteropoly acid structure include a method including modulating the pH of the slurry A in the range of 0.1 to 4 in step (ii) described above.

The lower limit and the upper limit of the pH of the slurry A are preferably 0.5 or more and 3 or less respectively. The structure of the dried catalyst product can be determined by infrared absorption spectrum analysis. In a case where the dried catalyst product has a Keggin-type heteropoly acid structure, an infrared absorption spectrum obtained has characteristic peaks at 1060, 960, 870, and 780 cm⁻¹.

<Step (vi)>

In step (vi), the dried catalyst product obtained in step (iii) is molded, to obtain a catalyst molded article. When the basic solution after washing the surface of the catalyst production apparatus in step (i) has a pH of 9 or more, favorable moldability is exhibited in step (iv).

The catalyst molded article obtained by molding is preferably further fired because performance is enhanced. Herein, such a catalyst molded article after molding and after firing is collectively referred to as “catalyst molded article”. Here, step (vi) may also be performed after a firing step described below.

### (Solvent)

The dried catalyst product is preferably mixed with a solvent and then molded from the viewpoint that the density of the catalyst molded article can be adjusted. The amount of the solvent used is appropriately selected depending on the type of the dried catalyst product, the particle shape, and the type of the solvent, and a decreased amount of the solvent used relative to the dried catalyst product leads to an increased molded article density of the resulting catalyst molded article and an increased amount of the solvent used relative to the dried catalyst product leads to a decreased molded article density of the resulting molded article. The amount of the solvent used based on 100 parts by mass of the dried catalyst product is preferably adjusted in the range of 10 to 70 parts by mass. When the amount of the solvent used based on 100 parts by mass of the dried catalyst product is 10 parts by mass or more, pores effective for production of α,β-unsaturated carboxylic acid in the resulting catalyst molded article tend to be increased. When the amount of the solvent used is 70 parts by mass or less, attachment during molding is reduced and handleability is enhanced. The lower limit and the upper limit of the amount of the solvent used based on 100 parts by mass of the dried catalyst product are preferably adjusted in the respective ranges of 15 parts by mass or more and 60 parts by mass or less.

The type of the solvent is not particularly limited, and water or an organic solvent is preferred. Examples of the organic solvent include lower alcohols such as methyl alcohol, ethanol, propyl alcohol, butyl alcohol and isopropanol, acetone, dimethyl ether, diethyl ether, methyl ethyl ketone, and ethyl acetate. These solvents may be used singly, or in combination of two or more kinds thereof. The solvent preferably includes at least the organic solvent.

### (Molding Aid)

Polyvinyl alcohol, polyvinyl alcohol, an α-glucan derivative, a β-glucan derivative, stearic acid, ammonium nitrate, graphite, or the like can be, if necessary, used as a molding aid during molding.

### (Molding Method)

The molding method of the dried catalyst product is not particularly limited. Examples include a molding method with any known extrusion molding machine, tablet molding machine, rolling granulator, or the like. In particular, a case of molding with an extrusion molding machine is preferred because the effects of the present embodiment can be sufficiently obtained. The extrusion molding machine here used can be, for example, an auger-type extrusion molding machine or a plunger-type extrusion molding machine, and a plunger-type extrusion molding machine is preferably used. The shape of the resulting catalyst molded article is not particularly limited, and examples include a ring shape, a columnar shape, a honeycomb shape, and a star shape.

In molding with the extrusion molding machine, an increased extrusion rate leads to an increased molded article density of the resulting catalyst molded article and a decreased extrusion rate leads to a decreased molded article density of the resulting catalyst molded article. The extrusion rate is preferably 50 to 500 mm/min. When the extrusion rate is 50 mm/min or more, the catalyst molded article can be stably produced. When the extrusion rate is 500 mm/min or less, pores effective for production of α,β-unsaturated carboxylic acid in the resulting catalyst molded article tend to be increased. The lower limit of the extrusion rate is more preferably 70 mm/min or more, further preferably 100 mm/min or more. The upper limit of the extrusion rate is more preferably 300 mm/min or less, further preferably 250 mm/min or less, particularly preferably 200 mm/min or less.

When the washing is performed in step (i) so that the basic solution after washing the surface of the catalyst production apparatus has a pH of 9 or more, the pressure rise in the extrusion molding machine in step (iii) is suppressed. Thus, pores effective for production of α,β-unsaturated carboxylic acid are kept, and the catalyst molded article, which provides a high yield of α,β-unsaturated carboxylic acid, is obtained. The reason for this is considered as follows.

In a case where the slurry A is produced with a catalyst production apparatus where a solid is attached onto the surface is used in step (ii), the solid serves as a core to promote particle growth of a heteropoly acid salt. Such a particle of a heteropoly acid salt, consequently formed, deteriorates the fluidity of the dried catalyst product in the extrusion molding machine. This causes clogging in the extrusion molding machine and leads to an increase in pressure in the extrusion molding machine. In this regard, when the washing is performed so that the basic solution after washing the surface of the catalyst production apparatus has a pH of 9 or more, the solid is sufficiently reduced, and thus a heteropoly acid salt particle having an appropriate particle size is formed in step (ii). Therefore, the dried catalyst product exhibits favorable fluidity in the extrusion molding machine, the pressure rise in the extrusion molding machine is suppressed, and the catalyst molded article, which has pores effective for production of α,β-unsaturated carboxylic acid, can be stably produced.

The maximum value of the pressure in the extrusion molding machine (hereinafter, also referred to as “maximum extrusion pressure”.) is preferably 15 MPa (G) or less, more preferably 12 MPa (G) or less, further preferably 10 MPa (G) or less, particularly preferably 5 MPa (G) or less.

The catalyst molded article obtained may be, if necessary, dried at 50 to 120° C., to remove a liquid.

### <Firing Step>

The catalyst molded article in step (iv) is preferably fired from the viewpoint of the yield of α,β-unsaturated carboxylic acid.

The firing can be performed under flow of at least one of an oxygen-containing gas such as air, and an inert gas, and the firing is preferably performed under flow of an oxygen-containing gas such as air. The inert gas here refers to a gas causing no reduction in catalyst activity, and examples include nitrogen, carbon dioxide gas, helium, and argon. These may be used singly, or as a mixture of two or more kinds thereof.

The shape of a firing container is not particularly limited, and, for example, a box-type or tubular container can be used. The firing can also be performed with division into a plurality of containers and filling. In particular, a tubular container having a cross-sectional area of 1 to 100 cm² is preferably used.

The firing temperature (the highest temperature during the firing) is preferably 200 to 700° C., and the lower limit is more preferably 320° C. or more and the upper limit is more preferably 450° C. or less.

As described above, a catalyst molded article can be produced. The production method can be used to stably obtain a catalyst molded article which is favorable in moldability and which provides a high yield of α,β-unsaturated carboxylic acid.

### [Method for Producing α,β-Unsaturated Carboxylic Acid]

A method for producing α,β-unsaturated carboxylic acid according to a second embodiment of the present invention is to produce α,β-unsaturated carboxylic acid by oxidation of α,β-unsaturated aldehyde in the presence of a catalyst molded article produced by the method of the above first embodiment.

Examples of the α,β-unsaturated aldehyde include (meth)acrolein, crotonaldehyde (β-methyl acrolein), and cinnamaldehyde (β-phenyl acrolein). In particular, (meth)acrolein is preferred and methacrolein is more preferred from the viewpoint of the yield of an objective product. The α,β-unsaturated carboxylic acid produced is α,β-unsaturated carboxylic acid in which an aldehyde group of the α,β-unsaturated aldehyde is converted into a carboxyl group. Specifically, in a case where the α,β-unsaturated aldehyde is (meth)acrolein, (meth)acrylic acid is obtained. Herein, the “(meth)acrolein” represents acrolein and methacrolein, and the “(meth)acrylic acid” represents acrylic acid and methacrylic acid.

Hereinafter, there is described, as a representative example, a method for producing methacrylic acid by oxidation of methacrolein in the presence of the catalyst molded article produced by the method according to the first embodiment.

In the method, methacrylic acid can be produced by contacting a raw material gas including methacrolein and oxygen, and the above catalyst molded article. This reaction can be usually performed in a fixed bed. The catalyst molded article layer may be a single layer, or may be of two or more layers. The catalyst molded article may be mixed with any other additive.

In the case of a fixed bed reactor, the location of the catalyst molded article layer in the reactor, the proportion of the catalyst molded article layer in the reactor, and the like are not particularly limited, and any form commonly used can be applied.

The concentration of methacrolein in the raw material gas is not particularly limited, and is preferably 1 to 20% by volume or more, and the lower limit is more preferably 3% by volume or more and the upper limit is more preferably 10% by volume or less. Any other component than methacrolein included in the raw material gas is not particularly limited, and examples include water, oxygen, and nitrogen. Here, methacrolein may include a small amount of impurities having no substantial influence on the present reaction of lower saturated aldehyde or the like.

The concentration of oxygen in the raw material gas based on 1 mol of methacrolein is preferably 0.4 to 4 mol. The lower limit of the concentration of oxygen is more preferably 0.5 mol or more, and the upper limit thereof is preferably 4 mol or less, more preferably 3 mol or less. The oxygen source is preferably air from the viewpoint of economic performance. If necessary, for example, an oxygen-rich gas may be used in which pure oxygen is added to the air.

The raw material gas may be one in which methacrolein and oxygen (or oxygen source) are diluted with an inert gas such as nitrogen or carbon dioxide. Water vapor may also be further added to the raw material gas. Methacrylic acid can be obtained at a high selectivity by a reaction performed in the presence of water vapor. The concentration of water vapor in the raw material gas is preferably 0.1 to 50% by volume, the lower limit is more preferably 18 by volume or more, and the upper limit is more preferably 40% by volume or less.

The contact time between the raw material gas and the catalyst molded article is preferably 0.1 to 30 seconds, the lower limit is more preferably 1.0 seconds or more, and the upper limit is more preferably 10 seconds or less.

The reaction pressure is preferably 0.1 to 1 MPa (G). Herein, (G) means the gauge pressure.

The reaction temperature is not particularly restricted and is preferably 200 to 450° C., the lower limit is more preferably 250° C. or more, and the upper limit is more preferably 400° C. or less.

### [Method for Producing α,β-Unsaturated Carboxylic Acid Ester]

A method for producing α,β-unsaturated carboxylic acid ester according to a third embodiment of the present invention includes esterifying α,β-unsaturated carboxylic acid produced by the method according to the second embodiment. According to such a method, α,β-unsaturated carboxylic acid ester can be obtained with α,β-unsaturated carboxylic acid obtained by oxidation of α,β-unsaturated aldehyde. The alcohol to be allowed to react with the α,β-unsaturated carboxylic acid is not particularly limited, and examples include methanol, ethanol, propanol, isopropanol, butanol, and isobutanol. Examples of the α,β-unsaturated carboxylic acid ester obtained include methyl (meth)acrylate, ethyl (meth)acrylate, propyl (meth)acrylate, isopropyl (meth)acrylate, butyl (meth)acrylate, and isobutyl (meth)acrylate. Such reaction can be performed in the presence of an acidic catalyst such as a sulfonic acid-type cation-exchange resin. The reaction temperature is preferably 50 to 200° C.

### EXAMPLES

Hereinafter, the present invention is specifically described with reference to Examples and Comparative Examples, but the present invention is not limited to these Examples. Herein, “part(s)” represents “part(s) by mass”.

### (Analysis of Solid)

The solid attached onto the surface of the catalyst production apparatus was confirmed with respect to each component by dissolving the solid in ammonia water and analyzing any dissolved component with ICP emission spectroscopy. The analysis apparatus here used was ICP Optima 8300 (manufactured by Perkin Elmer) at an output of 1300 W, a flow rate of plasma gas of 10 L/min, a flow rate of auxiliary gas of 0.2 L/min, and a flow rate of nebulizer gas of 0.55 L/min, with a division array type CCD as a detector.

### (pH of Basic Solution in Washing)

The pH of the basic solution after washing the surface of the catalyst production apparatus was measured with D-71 (manufactured by Horiba Ltd.) as a pH meter.

### (Compositional Ratio of Dried Catalyst Product)

The molar ratio of each element in the composition of the dried catalyst product was determined by dissolving the dried catalyst product in ammonia water and analyzing each dissolved component with ICP emission spectroscopy. The analysis apparatus here used was ICP Optima 8300 (manufactured by Perkin Elmer) at an output of 1300 W, a flow rate of plasma gas of 10 L/min, a flow rate of auxiliary gas of 0.2 L/min, and a flow rate of nebulizer gas of 0.55 L/min, with a division array type CCD as a detector. The molar ratio of an ammonium ion was determined by analyzing the dried catalyst product according to the Kjeldahl method.

### (Maximum Extrusion Pressure)

The pressure in the extrusion molding machine was measured with a pressure sensor inserted into a cylinder portion of the extrusion molding machine. Molding was performed 500 times under the same conditions, and the average was determined from the maximum value in the extrusion molding machine in each molding and defined as the maximum extrusion pressure.

### (Analysis of Raw Material Gas and Product)

The raw material gas and the product were analyzed with gas chromatography (apparatus: GC-2014 manufactured by Shimadzu Corporation, column: DB-FFAP manufactured by J&W, 30 m×0.32 mm, film thickness 1.0 μm). The yield of methacrylic acid was calculated from the results of gas chromatography, according to the following expression.

$$Yield of methacrylic acid ( \% ) = ( M 2 / M 1 ) \times 100$$

Here, M1 represents the number of moles of methacrolein fed and M2 represents the number of moles of methacrylic acid produced.

### Production Example 1

A preparation vessel provided with a rotor-stirring apparatus was used, 2000 parts of molybdenum trioxide, 108 parts of ammonium metavanadate, 160 parts of an aqueous 85% by mass phosphoric acid solution, and 56 parts of copper nitrate were dissolved in 8000 parts of pure water, and these were heated to 95° C. under stirring and stirred for 3 hours with the liquid temperature being kept at 95° C. After cooling to 40° C., a solution in which 270 parts of cesium bicarbonate was dissolved in 200 parts of pure water was added under stirring, and further stirred for 20 minutes, and thus a slurry was prepared.

The slurry obtained was dried with a spray dryer at a dryer inlet temperature of 300° C., and thus a dried product was obtained.

Ten parts of hydroxypropyl methylcellulose and 40 parts of ethanol were mixed with 200 parts of the dried product obtained, and kneaded by a kneading machine until a clayey product was obtained. Next, such a kneaded product obtained was molded with a plunger-type extrusion molding machine at an extrusion rate of 150 mm/min, and thus a columnar molded article having an outer diameter of 5.5 mm and an average length of 5.5 mm was obtained. The maximum extrusion pressure here and the standard deviation thereof are shown in Reference Example 1 in Table 1.

The molded article obtained was dried at 60° C. for 16 hours, and then fired under flow of air at 380° C. for 5 hours. The elemental composition excluding oxygen, of the resulting molded article after firing, was P_{1.2}Mo₁₂V_{0.8}Cu_{0.2}CS_{1.2}.

A reaction tube was filled with the resulting molded article, and a raw material gas including 5% by volume of methacrolein, 10% by volume of oxygen, 30% by volume of water vapor, and 55% by volume of nitrogen was allowed to flow thereinto for a contact time of 3.5 seconds, to perform oxidation reaction of methacrolein at a reaction temperature of 300° C. The results are shown in Reference Example 1 in Table 1.

Next, the same operation as that of the above slurry preparation was carried out in the same preparation vessel 50 times in total. Here, a solid was attached onto the inner wall of the preparation vessel used, and the rotor stirring apparatus used. It was confirmed with ICP emission spectroscopy that the solid included phosphorus, molybdenum, and cesium.

### Example 1

The preparation vessel where the solid was attached, used in Production Example 1, was loaded with pure water as the solvent in an amount of 0.95 times the volume of the preparation vessel, and heated to 50° C., and preliminary washing was performed with the liquid temperature being kept at 50° C. under stirring for 1 hour. After the preliminary washing, all the solvent used was discharged.

Next, the preparation vessel was loaded with an aqueous 4% by mass potassium hydroxide solution as the basic solution in an amount of 0.95 times the volume of the preparation vessel, and washed under stirring at a power required for stirring per unit volume of 4.0 kW/m³ at 25° C. and −0.002 MPa (G) for 3 hours. Here, such washing was performed under circulation with a circulation pump so that the linear velocity of such a basic aqueous solution circulated in the pipe connected to the catalyst production apparatus was 11 cm/s. The pH of the basic solution after the washing was 11.2. After the washing, all the basic solution used was discharged.

Next, pure water was loaded as the solvent in an amount of 0.95 times the volume of the preparation vessel, and heated to 50° C., and subsequent washing was performed under stirring at a power required for stirring of 4.0 kW/m³ with the liquid temperature being kept at 50° C. for 1 hour. The pH of the solvent after the subsequent washing was 6.7. After the subsequent washing, all the solvent used was discharged.

Next, the preparation vessel after such washing was used, 2000 parts of molybdenum trioxide, 108 parts of ammonium metavanadate, 160 parts of an aqueous 85% by mass phosphoric acid solution, and 56 parts of copper nitrate were dissolved in 8000 parts of pure water, and these were heated to 95° C. under stirring and stirred for 3 hours with the liquid temperature being kept at 95° C. After cooling to 40° C., a solution in which 270 parts of cesium bicarbonate was dissolved in 200 parts of pure water was added under stirring, and further stirred for 20 minutes, and thus a slurry A was prepared.

The slurry A obtained was dried with a spray dryer at a dryer inlet temperature of 300° C., and thus a dried catalyst product having a Keggin-type heteropoly acid structure was obtained.

Ten parts of hydroxypropyl methylcellulose and 40 parts of ethanol were mixed with 200 parts of the dried catalyst product obtained, and kneaded by a kneading machine until a clayey product was obtained. Next, such a kneaded product obtained was molded with a plunger-type extrusion molding machine at an extrusion rate of 150 mm/min, and thus a columnar catalyst molded article having an outer diameter of 5.5 mm and an average length of 5.5 mm was obtained. The maximum extrusion pressure here and the standard deviation thereof are shown in Table 1.

The catalyst molded article obtained was dried at 60° C. for 16 hours, and then fired under flow of air at 380° C. for 5 hours. The elemental composition excluding oxygen, of the resulting catalyst molded article after firing, was P_{1.2}Mo₁₂V_{0.8}Cu_{0.2}CS_{1.2}.

A reaction tube was filled with the resulting catalyst molded article, and a raw material gas including 5% by volume of methacrolein, 10% by volume of oxygen, 30% by volume of water vapor, and 55% by volume of nitrogen was allowed to flow thereinto for a contact time of 3.5 seconds, to perform oxidation reaction of methacrolein at a reaction temperature of 300° C. The results are shown in Table 1.

### Example 2

The preparation vessel where the solid was attached, obtained by the same method as in Production Example 1, was subjected to preliminary washing carried out by the same method as in Example 1. After the preliminary washing, all the solvent used was discharged.

Next, washing was carried out by the same method as in Example 1 except that an aqueous 2% by mass potassium hydroxide solution was used as the basic solution. The pH of the basic solution after the washing was 10.0. After the washing, all the basic solution used was discharged.

Next, subsequent washing was carried out by the same method as in Example 1. The pH of the solvent after the subsequent washing was 6.8. After the subsequent washing, all the solvent used was discharged.

Next, the preparation vessel after such washing was used, and a slurry A was prepared by the same method as in Example 1.

The slurry A obtained was dried by the same method as in Example 1, and thus a dried catalyst product having a Keggin-type heteropoly acid structure was obtained.

The dried catalyst product obtained was molded by the same method as in Example 1, and thus a columnar catalyst molded article having an outer diameter of 5.5 mm and an average length of 5.5 mm was obtained. The maximum extrusion pressure here and the standard deviation thereof are shown in Table 1.

The catalyst molded article obtained was dried and fired by the same method as in Example 1. The elemental composition excluding oxygen, of the resulting catalyst molded article fired, was P_{1.2}Mo₁₂V_{0.8}Cu_{0.2}CS_{1.2}.

A reaction tube was filled with the catalyst molded article obtained, and oxidation reaction of methacrolein was performed by the same method as in Example 1. The results are shown in Table 1.

### Example 3

The preparation vessel where the solid was attached, obtained by the same method as in Production Example 1, was subjected to preliminary washing performed by the same method as in Example 1. After the preliminary washing, all the solvent used was discharged.

Next, washing was performed by the same method as in Example 1. The pH of the basic solution after the washing was 11.2. After the washing, all the basic solution used was discharged.

Next, subsequent washing was performed by the same method as in Example 1. The pH of the solvent after the subsequent washing was 6.7. After the subsequent washing, all the solvent used was discharged.

Next, the preparation vessel after the subsequent washing was used, and a slurry A was prepared by the same method as in Example 1.

The slurry A obtained was dried by the same method as in Example 1, and thus a dried catalyst product having a Keggin-type heteropoly acid structure was obtained.

The dried catalyst product obtained was molded by the same method as in Example 1 except that the extrusion rate was 400 mm/min, and thus a columnar catalyst molded article having an outer diameter of 5.5 mm and an average length of 5.5 mm was obtained. The maximum extrusion pressure here and the standard deviation thereof are shown in Table 1.

The catalyst molded article obtained was dried and fired by the same method as in Example 1. The elemental composition excluding oxygen, of the resulting catalyst molded article fired, was P_{1.2}Mo₁₂V_{0.8}Cu_{0.2}CS_{1.2}.

A reaction tube was filled with the catalyst molded article obtained, and oxidation reaction of methacrolein was performed by the same method as in Example 1. The results are shown in Table 1.

### Example 4

The preparation vessel where the solid was attached, obtained by the same method as in Production Example 1, was subjected to preliminary washing performed by the same method as in Example 1. After the preliminary washing, all the solvent used was discharged.

Next, the preparation vessel was loaded with an aqueous 4% by mass potassium hydroxide solution as the basic solution in an amount of 0.95 times the volume of the preparation vessel at 25° C. and −0.002 MPa (G) for 3 hours. The basic solution was not here stirred and circulated. The pH of the basic solution after the washing was 11.7. After the washing, all the basic solution used was discharged.

Next, subsequent washing was carried out by the same method as in Example 1. The pH of the solvent after the subsequent washing was 6.8. After the subsequent washing, all the solvent used was discharged.

Next, the preparation vessel after the subsequent washing was used, and a slurry A was prepared by the same method as in Example 1.

The slurry A obtained was dried by the same method as in Example 1, and thus a dried catalyst product having a Keggin-type heteropoly acid structure was obtained. The dried catalyst product obtained was molded by the same method as in Example 1, and thus a columnar catalyst molded article having an outer diameter of 5.5 mm and an average length of 5.5 mm was obtained. The maximum extrusion pressure here and the standard deviation thereof are shown in Table 1.

The catalyst molded article obtained was dried and fired by the same method as in Example 1. The elemental composition excluding oxygen, of the resulting catalyst molded article fired, was P_{1.2}Mo₁₂V_{0.8}Cu_{0.2}CS_{1.2}.

A reaction tube was filled with the catalyst molded article obtained, and oxidation reaction of methacrolein was performed by the same method as in Example 1. The results are shown in Table 1.

### Comparative Example 1

The preparation vessel where the solid was attached, obtained by the same method as in Production Example 1, was loaded with pure water as the solvent in an amount of 0.95 times the volume of the preparation vessel, heated to 50° C., and washed under stirring at a power required for stirring per unit volume of 4.0 kW/m³ at 50° C. and −0.002 MPa (G) for 3 hours. The pH of the solvent after such washing was 6.1. After such washing, all the solvent used was discharged.

Next, the preparation vessel after such washing was used, and a slurry A was prepared by the same method as in Example 1.

The slurry A obtained was dried by the same method as in Example 1, and thus a dried catalyst product having a Keggin-type heteropoly acid structure was obtained.

The dried catalyst product obtained was molded by the same method as in Example 1, and thus a columnar catalyst molded article having an outer diameter of 5.5 mm and an average length of 5.5 mm was obtained. The maximum extrusion pressure here and the standard deviation thereof are shown in

The catalyst molded article obtained was dried and fired by the same method as in Example 1. The elemental composition excluding oxygen, of the resulting catalyst molded article fired, was P_{1.2}Mo₁₂V_{0.8}Cu_{0.2}CS_{1.2}.

A reaction tube was filled with the catalyst molded article obtained, and oxidation reaction of methacrolein was performed by the same method as in Example 1. The results are shown in Table 1.

### Comparative Example 2

The preparation vessel where the solid was attached, obtained by the same method as in Production Example 1, was subjected to preliminary washing carried out by the same method as in Example 1. After the preliminary washing, all the solvent used was discharged.

Next, washing was carried out by the same method as in Example 1 except that an aqueous 0.5% by mass potassium hydroxide solution was used as the basic solution. The pH of the basic solution after the washing was 8.4. After the washing, all the basic solution used was discharged.

Next, subsequent washing was carried out by the same method as in Example 1. The pH of the solvent after the subsequent washing was 6.7. After the subsequent washing, all the solvent used was discharged.

Next, the preparation vessel after such washing was used, and a slurry A was prepared by the same method as in Example 1.

The slurry A obtained was dried by the same method as in Example 1, and thus a dried catalyst product having a Keggin-type heteropoly acid structure was obtained.

The dried catalyst product obtained was molded by the same method as in Example 1, and thus a columnar catalyst molded article having an outer diameter of 5.5 mm and an average length of 5.5 mm was obtained. The maximum extrusion pressure here and the standard deviation thereof are shown in Table 1.

The catalyst molded article obtained was dried and fired by the same method as in Example 1. The elemental composition excluding oxygen, of the resulting catalyst molded article fired, was P_{1.2}Mo₁₂V_{0.8}Cu_{0.2}CS_{1.2}.

A reaction tube was filled with the catalyst molded article obtained, and oxidation reaction of methacrolein was performed by the same method as in Example 1. The results are shown in Table 1.

### <Comparative Example 3>

The preparation vessel where the solid was attached, obtained by the same method as in Production Example 1, was subjected to preliminary washing carried out by the same method as in Example 1. After the preliminary washing, all the solvent used was discharged.

Next, washing was carried out by the same method as in Example 1 except that an aqueous 4% by mass ammonium carbonate solution was used as the basic solution. The pH of the basic solution after the washing was 7.9. After the washing, all the basic solution used was discharged.

Next, subsequent washing was carried out by the same method as in Example 1. The pH of the solvent after the subsequent washing was 6.8. After the subsequent washing, all the solvent used was discharged.

Next, the preparation vessel after such washing was used, and a slurry A was prepared by the same method as in Example 1.

The slurry A obtained was dried by the same method as in Example 1, and thus a dried catalyst product having a Keggin-type heteropoly acid structure was obtained.

The dried catalyst product obtained was molded by the same method as in Example 1, and thus a columnar catalyst molded article having an outer diameter of 5.5 mm and an average length of 5.5 mm was obtained. The maximum extrusion pressure here and the standard deviation thereof are shown in

The catalyst molded article obtained was dried and fired by the same method as in Example 1. The elemental composition excluding oxygen, of the resulting catalyst molded article fired, was P_{1.2}Mo₁₂V_{0.8}Cu_{0.2}CS_{1.2}.

A reaction tube was filled with the catalyst molded article obtained, and oxidation reaction of methacrolein was performed by the same method as in Example 1. The results are shown in Table 1.

### Production Example 2

The same preparation vessel as in Production Example 1 was used, and 4000 parts of pure water, 2000 parts of ammonium paramolybdate, 24.8 parts of ammonium paratungstate, 5.6 parts of potassium nitrate, 110 parts of antimony trioxide and 198 parts of bismuth trioxide were added thereto, and heated to 50° C. (liquid A). Separately, 495.6 parts of ferric nitrate, 1153.6 parts of cobalt nitrate and 140.4 parts of zinc nitrate were sequentially added to and dissolved in 4000 parts of pure water (liquid B). Next, the liquid B was added with the liquid A being stirred, and thus an aqueous slurry was obtained. The aqueous slurry was heated to 95° C. under stirring, and stirred for 90 minutes. Thereafter, the aqueous slurry was heated to 103° C. for 1 hour, and thus a slurry was obtained.

The slurry obtained was dried with a spray dryer at a dryer inlet temperature of 300° C., and thus a dried product was obtained.

The dried product obtained was calcined at 300° C. for 1 hour, and furthermore fired at 500° C. for 3 hours, and thus a fired powder was obtained.

Six parts of methylcellulose and 72 parts of pure water were mixed with 200 parts of the fired powder obtained, and kneaded by a kneading machine until a clayey product was obtained. Next, such a kneaded product obtained was molded with a plunger-type extrusion molding machine at an extrusion rate of 150 mm/min, and thus a ring-shaped molded article having an outer diameter of 5 mm, an inner diameter of 2 mm, and an average length of 5 mm was obtained. The maximum extrusion pressure here and the standard deviation thereof are shown in Reference Example 2 in Table 2.

The molded article obtained was dried at 110° C. for 16 hours, and then fired under flow of air at 510° C. for 3 hours. The elemental composition excluding oxygen, of the resulting molded article after firing, was Mo₁₂W_{0.1}Bi_{0.9}Fe_{1.3}Sb_{0.8}Co_{4.2}Zn_{0.5}K_{0.06}.

Next, the same operation as that of the above slurry preparation was carried out in the same preparation vessel 5 times in total. Here, a solid was attached onto the inner wall of the preparation vessel used, and the rotor stirring apparatus used. It was confirmed with ICP emission spectroscopy that the solid included molybdenum, bismuth and iron.

### Reference Example 3

The preparation vessel where the solid was attached, used in Production Example 2, was subjected to preliminary washing carried out by the same method as in Example 1. After the preliminary washing, all the solvent used was discharged.

Next, washing was performed by the same method as in Example 1 except that an aqueous 4% by mass potassium hydroxide solution was used as the basic solution. The pH of the basic solution after the washing was 11.6. After the washing, all the basic solution used was discharged.

Next, subsequent washing was performed by the same method as in Example 1. The pH of the solvent after the subsequent washing was 6.7. After the subsequent washing, all the solvent used was discharged.

Next, the preparation vessel after such washing was used, and 4000 parts of pure water, 2000 parts of ammonium paramolybdate, 24.8 parts of ammonium paratungstate, 5.6 parts of potassium nitrate, 110 parts of antimony trioxide and 198 parts of bismuth trioxide were added thereto, and heated to 50° C. (liquid A). Separately, 495.6 parts of ferric nitrate, 1153.6 parts of cobalt nitrate and 140.4 parts of zinc nitrate were sequentially added to and dissolved in 4000 parts of pure water (liquid B). The liquid B was added with the liquid A being stirred, and thus an aqueous slurry was obtained. The aqueous slurry was heated to 95° C. under stirring, and stirred for 90 minutes. Thereafter, the aqueous slurry was heated to 103° C. for 1 hour, and thus a slurry was obtained.

The slurry obtained was dried with a spray dryer at a dryer inlet temperature of 300° C., and thus a dried product was obtained.

The dried product obtained was calcined at 300° C. for 1 hour, and furthermore fired at 500° C. for 3 hours, and thus a fired powder was obtained.

Six parts of methylcellulose and 72 parts of pure water were mixed with 200 parts of the fired powder obtained, and kneaded by a kneading machine until a clayey product was obtained. Next, such a kneaded product obtained was molded with a plunger-type extrusion molding machine at an extrusion rate of 150 mm/min, and thus a ring-shaped molded article having an outer diameter of 5 mm, an inner diameter of 2 mm, and an average length of 5 mm was obtained. The maximum extrusion pressure here and the standard deviation thereof are shown in Table 2.

The molded article obtained was dried at 110° C. for 16 hours, and then fired under flow of air at 510° C. for 3 hours.

The elemental composition excluding oxygen, of the resulting molded after article firing, was Mo₁₂W_{0.1}Bi_{0.9}Fe_{1.3}Sb_{0.8}Co_{4.2}Zn_{0.5}K_{0.06}.

A reaction tube was filled with the resulting molded article, and a raw material gas including 5% by volume of isobutylene, 12% by volume of oxygen, 10% by volume of water vapor, and 73% by volume of nitrogen was allowed to flow thereinto for a contact time of 3.6 seconds, to perform oxidation reaction of isobutylene at a reaction temperature of 340° C. The results are shown in Table 2.

### Reference Example 4

The preparation vessel where the solid was attached, obtained by the same method as in Production Example 2, was loaded with pure water as the solvent in an amount of 0.95 times the volume of the preparation vessel, heated to 50° C., and washed under stirring at a power required for stirring per unit volume of 4.0 kW/m³ at 50° C. and −0.002 MPa (G) for 3 hours. The pH of the solvent after such washing was 6.2. After such washing, all the solvent used was discharged.

Next, the preparation vessel after such washing was used and a slurry was prepared by the same method as in Reference Example 3.

The slurry obtained was dried by the same method as in Reference Example 3, and thus a dried product was obtained.

The dried product obtained was calcined and fired by the same method as in Reference Example 3, and thus a fired powder was obtained.

The fired powder obtained was molded by the same method as in Reference Example 3, and thus a ring-shaped molded article having an outer diameter of 5 mm, an inner diameter of 2 mm, and an average length of 5 mm was obtained. The maximum extrusion pressure here and the standard deviation thereof are shown in Table 2.

The molded article obtained was dried and fired by the same method as in Reference Example 3. The elemental composition excluding oxygen, of the resulting molded article after firing, was Mo₁₂W_{0.1}Bi_{0.9}Fe_{1.3}Sb_{0.8}Co_{4.2}Zn_{0.5}K_{0.06}.

A reaction tube was filled with the resulting molded article, and oxidation reaction of isobutylene was performed by the same method as in Reference Example 3. The results are shown in Table 2.

TABLE 1

| | | | | Standard | |
|---|---|---|---|---|---|
| | | pH of | Maximum | deviation of | Yield of |
| | | washing | extrusion | maximum | methacrylic |
| | | liquid after | pressure | extrusion | acid |
| | Washing liquid | washing | [MPa (G)] | pressure | [%] |
| Reference | — | — | 4.5 | 0.8 | 49.9 |
| Example 1 | | | | | |
| Example 1 | Aqueous 4% by mass | 11.2 | 4.7 | 1.0 | 50.1 |
| | potassium hydroxide solution | | | | |
| Example 2 | Aqueous 2% by mass | 10.0 | 4.8 | 1.1 | 49.9 |
| | potassium hydroxide solution | | | | |
| Example 3 | Aqueous 4% by mass | 11.2 | 5.5 | 1.4 | 49.5 |
| | potassium hydroxide solution | | | | |
| Example 4 | Aqueous 4% by mass | 11.7 | 5.0 | 1.3 | 49.7 |
| | potassium hydroxide solution | | | | |
| Comparative | Pure water | 6.1 | 6.4 | 2.0 | 48.1 |
| Example 1 | | | | | |
| Comparative | Aqueous 0.5% by mass | 8.4 | 5.8 | 1.9 | 48.5 |
| Example 2 | potassium hydroxide solution | | | | |
| Comparative | Aqueous 4% by mass | 7.9 | 6.0 | 1.9 | 48.4 |
| Example 3 | ammonium carbonate solution | | | | |

TABLE 2

| | | | | Standard |
|---|---|---|---|---|
| | | pH of | Maximum | deviation of |
| | | washing | extrusion | maximum |
| | | liquid after | pressure | extrusion |
| | Washing liquid | washing | [MPa (G)] | pressure |
| Reference | — | — | 5.6 | 0.9 |
| Example 2 | | | | |
| Reference | Aqueous 4% by | 11.6 | 5.6 | 0.9 |
| Example 3 | mass potassium | | | |
| | hydroxide solution | | | |
| Reference | Pure water | 6.2 | 5.7 | 0.9 |
| Example 4 | | | | |

As shown in Table 1, each of Examples 1 and 2, in which a slurry A was prepared with a preparation vessel washed under specified conditions, allowed for the maximum extrusion pressure stably kept at a low value in molding of a dried catalyst product. Thus, it can be said that a catalyst molded article can be obtained in which pores effective for production of methacrylic acid are maintained and the yield of methacrylic acid is high.

In this regard, as shown in Table 2, washing conditions of a preparation vessel were not confirmed to have any influence on molding of a dried product in a case of no inclusion of any step of preparing a slurry A containing a heteropoly acid salt including phosphorus and molybdenum.

Here, methacrylic acid obtained in the present Examples can be esterified, thereby obtaining methacrylic acid ester.

### INDUSTRIAL APPLICABILITY

According to the present invention, a catalyst molded article which can allow a high yield of α,β-unsaturated carboxylic acid to be achieved in production of α,β-unsaturated carboxylic acid can be stably provided, and therefore industrial usefulness is obtained.

What is claimed is:

1. A method for producing a catalyst molded article for use in the production of α,β-unsaturated carboxylic acid, by oxidation of α,β-unsaturated aldehyde, the method comprising
(i) a step of washing a surface of a catalyst production apparatus where a solid including phosphorus, molybdenum and an alkali metal is attached onto the surface, with a basic solution,
(ii) a step of using the catalyst production apparatus, to prepare a slurry A containing a heteropoly acid salt including phosphorus and molybdenum,
(iii) a step of drying the slurry A, to obtain a dried catalyst product, and
(iv) a step of molding the dried catalyst product, to obtain a catalyst molded article, wherein the basic solution after washing the surface of the catalyst production apparatus in step (i) has a pH of 9 or more.

2. The method for producing a catalyst molded article according to claim 1, wherein the dried catalyst product is molded with an extrusion molding machine in step (iv).

3. The method for producing a catalyst molded article according to claim 2, wherein the dried catalyst product is molded at an extrusion rate of 50 to 500 mm/min in step (iv).

4. The method for producing a catalyst molded article according to claim 2, wherein the dried catalyst product is molded at an extrusion rate of 50 to 300 mm/min in step (iv).

5. The method for producing a catalyst molded article according to claim 2, wherein the maximum value of the pressure in the extrusion molding machine in step (iv) is 15 MPa or less in terms of gauge pressure.

6. The method for producing a catalyst molded article according to claim 1, wherein the surface of the catalyst production apparatus is washed with the basic solution being stirred at a power required for stirring per unit volume of 0.01 to 6 kW/m³ in step (i).

7. The method for producing a catalyst molded article according to claim 1, wherein the surface of the catalyst production apparatus is washed with the basic solution being circulated in a pipe connected to the catalyst production apparatus, by use of a circulation pump, in step (i).

8. The method for producing a catalyst molded article according to claim 7, wherein the linear velocity of the basic solution circulated in the pipe is 1 to 100 cm/s.

9. The method for producing a catalyst molded article according to claim 1, wherein the dried catalyst product has a composition represented by the following formula (I):
PₐMO_{b}V_{c}CU_{d}AₑE_{f}G_{g}(NH₄)ₕOᵢ (I)
wherein P, Mo, V, Cu, NH₄ and 0 respectively represent phosphorus, molybdenum, vanadium, copper, ammonium radical and oxygen, A represents at least one element selected from the group consisting of antimony, bismuth, arsenicum, germanium, tellurium, selenium, silicon and tungsten, E represents at least one element selected from the group consisting of iron, zinc, chromium, tantalum, cobalt, nickel, manganese, titanium and niobium, G represents at least one element selected from the group consisting of potassium, rubidium and cesium, a to i each represent the molar ratio of each component, b=12, a =0.5 to 3, c=0.01 to 3, d=0.01 to 2, e=0 to 3, f=0 to 3, g=0.01 to 3, and h=0 to 20 are satisfied, and i is the molar ratio of oxygen necessary for satisfying the valence of such each component.

10. The method for producing a catalyst molded article according to claim 1, wherein the dried catalyst product has a Keggin-type heteropoly acid structure.

11. The method for producing a catalyst molded article according to claim 1, comprising a step of washing the surface of the catalyst production apparatus, washed with the basic solution in step (i), with water, to remove the basic solution.

12. The method for producing a catalyst molded article according to claim 11, wherein the basic solution is removed by washing with stirring of water at a power required for stirring per unit volume of 0.01 to 6 kW/m³.

13. A method for producing α,β-unsaturated carboxylic acid, comprising oxidizing α,β-unsaturated aldehyde in the presence of a catalyst molded article produced by the method according to claim 1.

14. A method for producing α,β-unsaturated carboxylic acid ester, comprising esterifying α,β-unsaturated carboxylic acid produced by the method according to claim 13.
